(19) United States
(12) Patent Application Publication
(10) Pub. No.: US 2025/0102438 A1
(43) Pub. Date: Mar. 27, 2025

(54) INTRINSIC FLUORESCENCE GENERATION USING EXOGENOUS FLUORESCENCE AGENT

(71) Applicant: MediBeacon Inc., St. Louis, MO (US)

(72) Inventors: Jesse Ross-Jones, Wittlich (DE); Jochen Friedemann, Altrip (DE)

(21) Appl. No.: 18/897,554

(22) Filed: Sep. 26, 2024

Related U.S. Application Data
(60) Provisional application No. 63/540,510, filed on Sep. 26, 2023.

(51) Int. Cl. G01N 21/64

(52) U.S. Cl. CPC G01N 21/6486; G01N 21/6408

(57) ABSTRACT

Method, system, and apparatus determines glomerular filtration rate (GFR) by obtaining a measurement data set. Furthermore, the disclosure includes obtaining a measurement data set comprising a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; determining if the IF signal includes a portion of premature fluorescence data; identifying, upon determination of the portion of premature fluorescence data, an operable IF signal range by filtering the IF signal associated with premature fluorescence data; determining a GFR value in the patient based on a rate of change of the operable IF signal; providing the GFR value.

### CROSS-REFERENCE TO RELATED APPLICATION

This application claims priority to U.S. Provisional Application No. 63/540,510, filed Sep. 26, 2023 which is hereby incorporated by reference in its entirety.

### FIELD

The present disclosure relates generally to an operable intrinsic fluorescence signal including adjusting based upon premature fluorescence data.

### BACKGROUND

Monitoring of a biological parameter in patients that are critically ill or injured is important. Organ function can be impaired due to organ damage, aging, an underlying illness, and others. Assessing organ function allows for determination of an organ damage and/or organ failure.

### BRIEF DESCRIPTION OF THE DRAWINGS

Details of one or more aspects of the subject matter described in this disclosure are set forth in the accompanying drawings and the description below. However, the accompanying drawings illustrate only some typical aspects of this disclosure and are therefore not to be considered limiting of its scope. Other features, aspects, and advantages will become apparent from the description, the drawings and the claims.

In order to describe the manner in which the above-recited and other advantages and features of the disclosure can be obtained, a more particular description of the principles briefly described above will be rendered by reference to specific examples thereof which are illustrated in the appended drawings. Understanding that these drawings depict only exemplary examples of the disclosure and are not, therefore, to be considered to be limiting of its scope, the principles herein are described and explained with additional specificity and detail through the use of the accompanying drawings in which:

FIG. 1 is a schematic illustration of a single light source monitoring device in one aspect;

FIG. 2 illustrates illumination from a light source and exogenous fluorescence agent, and detection by a first detector and a second detector;

FIG. 3 illustrates illumination of light from another light source and detection by a first detector and a second detector;

FIG. 4 is a schematic illustration of a multi-light source monitoring system in one aspect;

FIG. 5 illustrates an example of a sensor head having one or more sources and two detectors;

FIG. 6 is an exploded view of the inner housing of the sensor head illustrated in FIG. 5;

FIG. 7 is a graph summarizing the absorption, transmission, and emission spectra of various devices, materials, and compounds associated with the non-invasive monitoring of an exogenous fluorescent agent in vivo defined over light wavelengths ranging from about 430 nm to about 650 nm;

FIG. 8 is a graph of representative intrinsic fluorescence signal measurements (IF_{agent}) detected by a renal monitoring device obtained before and after administration of an exogenous fluorescent agent;

FIG. 9 illustrates a flow chart corresponding to method of determining glomerular filtration rate (GFR) with an exogenous fluorescent agent.

### DETAILED DESCRIPTION

Various examples of the disclosure are discussed in detail below. While specific implementations are discussed, it should be understood that this is done for illustration purposes only. A person skilled in the relevant art will recognize that other components and configurations can be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an example in the present disclosure can be references to the same example or any example; and, such references mean at least one of the examples.

Reference to “one example” or “an example” means that a particular feature, structure, or characteristic described in connection with the example is included in at least one example of the disclosure. The appearances of the phrase “in one example” in various places in the specification are not necessarily all referring to the same example, nor are separate or alternative examples mutually exclusive of other examples. Moreover, various features are described which can be exhibited by some examples and not by others.

The terms used in this specification generally have their ordinary meanings in the art, within the context of the disclosure, and in the specific context where each term is used. Alternative language and synonyms can be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various examples given in this specification.

Without intent to limit the scope of the disclosure, examples of instruments, apparatus, methods and their related results according to the examples of the present disclosure are given below. Note that titles or subtitles can be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions, will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein. Additionally, unless specifically required the order of one or more of the steps can be as described or the order can be adapted.

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

The phrase “diffuse reflecting medium” refers to any material through which light propagates, which includes a plurality of moieties, particles, or molecules that can scatter, reflect, and/or absorb the light as it propagates. The distribution of the plurality of moieties, particles, and/or molecules can be uniform or non-uniform and can change over time.

The present disclosure provides non-invasive monitoring of a biological parameter indicative of organ function in a patient based upon an intrinsic fluorescence (IF) signal. The present disclosure makes use of a unique filtering of the IF signal to determine if the IF signal includes a portion of premature fluorescence data and identifies, upon determination of the portion of premature fluorescence data, an operable IF signal range by filtering the IF signal associated with premature fluorescence data. The IF signal is based upon use of a suitable indicator that is administered to a patient. In one example, the administration to the patient can be through injection. In another example, the administration to the patient can be through ingestion. In one example, the organ function is intestinal wall barrier function wherein the intestinal wall barrier function in a patient is assessed based upon an IF signal. In at least one example, the organ function is renal function. The present disclosure increases accuracy of calculating the renal function in a patient based upon an IF signal.

Suitable indicator substances for use with the methods and devices described herein are disclosed in U.S. Pat. Nos. 8,155,000, 8,664,392, 8,697,033, 8,703,100, 8,722,685, 8,778,309, 9,005,581, 9,283,288, 9,376,399, RE47,413, RE47,255, 10,137,207, 10,525,149, and 11,590,244, which are all incorporated by reference in their entirety for all purposes. In some aspects, the indicator substance is eliminated from the body of a patient by glomerular filtration. In some aspects, the indicator substance is eliminated from the body of a patient only by glomerular filtration. In some aspects, the indicator substance is a GFR agent.

Disclosed are systems, apparatuses, methods, computer readable medium, and circuits for monitoring a biological parameter indicative of organ function using an exogenous fluorescent agent in a patient. According to at least one example, the method includes: obtaining a measurement data set comprising a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; determining if the IF signal includes a portion of premature fluorescence data; identifying, upon determination of the portion of premature fluorescence data, an operable IF signal range by filtering the IF signal associated with premature fluorescence data; determining a biological parameter value in the patient based on a rate of change of the operable IF signal; providing the biological parameter value. For example, the system obtains a measurement data set comprising a plurality of measurement entries that are obtained before and after administration of an exogenous fluorescent agent; generates an intrinsic fluorescence (IF) signal representing a detected fluorescence intensity emitted by the exogenous fluorescent agent from within a diffuse reflecting medium; determines if the IF signal includes a portion of premature fluorescence data; identifies, upon determination of the portion of premature fluorescence data, an operable IF signal range by filtering the IF signal associated with premature fluorescence data; determines a biological parameter value in the patient based on a rate of change of the operable IF signal; providing the biological parameter value.

In another example, a system for determining a biological parameter, e.g. glomerular filtration rate (GFR), using an exogenous fluorescent agent in a patient is provided that includes a storage (e.g., a memory configured to store data, such as virtual content data, one or more images, etc.) and one or more processors (e.g., implemented in circuitry) coupled to the memory and configured to execute instructions and, in conjunction with various components (e.g., a network interface, a display, an output device, etc.), cause the system to: obtain a measurement data set comprising a plurality of measurement entries that are obtained before and after administration of an exogenous fluorescent agent; generate an intrinsic fluorescence (IF) signal representing a detected fluorescence intensity emitted by the exogenous fluorescent agent from within a diffuse reflecting medium; determine if the IF signal includes a portion of premature fluorescence data; identifies, upon determination of the portion of premature fluorescence data, an operable IF signal range by filtering the IF signal associated with premature fluorescence data; determine a biological parameter value, such as a glomerular filtration rate (GFR) value, in the patient based on a rate of change of the operable IF signal; providing the biological parameter (for example, GFR) value.

According to another example, a method for determining intestinal barrier wall function includes: obtaining a measurement data set comprising a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; determining if the IF signal includes a portion of premature fluorescence data; identifying, upon determination of the portion of premature fluorescence data, an operable IF signal range by filtering the IF signal associated with premature fluorescence data; determining a value of intestinal barrier wall function in the patient based on a rate of change of the operable IF signal; providing the intestinal barrier wall function value.

In another example, the system for determining a biological parameter described above determines intestinal barrier wall function by determining a value of intestinal barrier wall function in the patient based on the rate of change of the operable IF signal thereby providing the intestinal barrier wall function value.

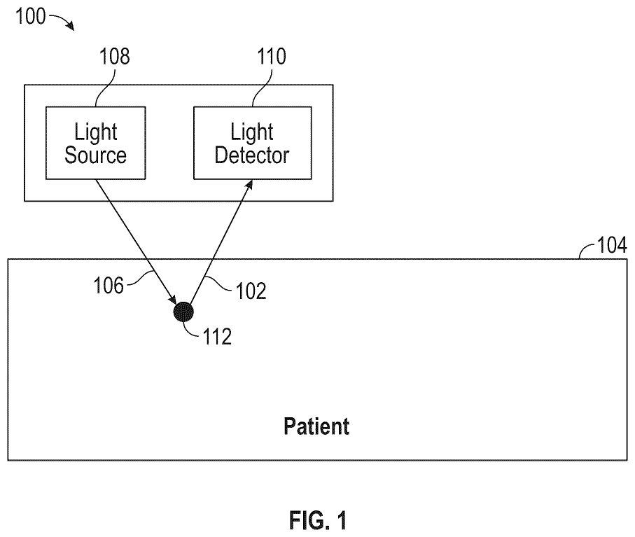

FIG. 1 is a schematic illustration of a system 100 in which an exogenous fluorescent agent 112 is administered to a patient. A light source 108 emits light 106 into the patient 104. The light 106 can be described as an excitation light or an excitation light source. The light 106 can also be controlled to be at one wavelength, multiple wavelengths that vary over time, or multiple wavelengths emitted simultaneously. The exogenous fluorescent agent 112 produces fluorescence 102 in response to an excitation event including: illumination by light 106 at an excitation wavelength (λₑₓ), occurrence of an enzymatic reaction, changes in local electrical potential, and any other known excitation event associated with exogenous fluorescent agents. The light source 108 can be configured to deliver light 106 at an excitation wavelength (λₑₓ) to the patient 104.

Fluorescence 102 can produce as described above. In at least one example, the excitation wavelength (λₑₓ) of the light 106 and the emission wavelength (λₑₘ) of the fluorescence 102 can be spectrally distinct (i.e., (λₑₓ) is sufficiently different from (λₑₘ) so that the light detector 110 can be configured to selectively detect only the fluorescence 102 by the inclusion of any known optical wavelength separation device including an optical filter).

Change in the fluorescence 102 can be analyzed to obtain information regarding organ function of the patient 104. As described herein, two non-limiting examples of organ function can be one of renal function and/or intestinal wall barrier function. In one example, the rate of decrease in fluorescence 102 can be proportional to the rate of removal of the exogenous fluorescent agent 112 by one or more organs of the patient 104, thereby providing a biological parameter value. In another non-limiting example, the rate of decrease in fluorescence 102 can be proportional to the rate of removal of the exogenous fluorescent agent 112 by the kidneys of the patient 104, thereby providing a measurement of renal function including: renal decay time constant (RDTC) and/or glomerular filtration rate (GFR). Additionally, the present disclosure can calculate a permeability or leak measurement when the functioning of the intestines is measured.

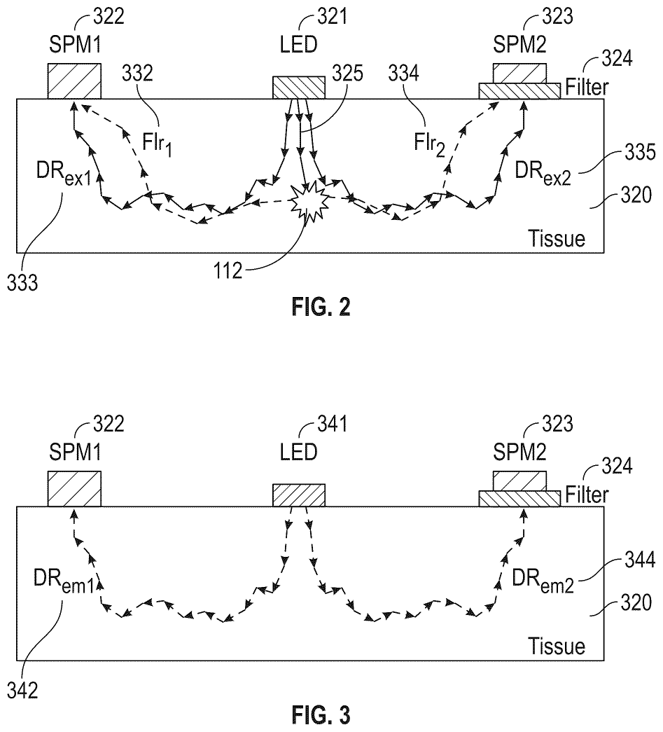

FIG. 2 illustrates using a light source 108 in the form of an excitation light emitting diode (LED) 321 in the presence of the exogenous fluorescent agent 112. The excitation LED 321 can emit light at one or more different excitation wavelengths. In one example, the excitation light wavelength can be a blue light. In other examples, the excitation light wavelength can be a blue light and a green light. In other examples, the excitation light wavelength can be chosen based upon the selected exogenous fluorescent agent 112. The light detector 110 can be in the form of a first light detector 322 and a second light detector 323. As illustrated the first light detector 322 can receive a first signal labeled SPM1 and the second light detector 323 can receive a second signal labeled SPM2. In at least one example, the first light detector 322 and the second light detector 323 can be a silicon photomultiplier. While the light detectors 322, 323 can take a varied of different forms, the silicon photomultiplier and/or photo diodes can provide for desired characteristics to perform the measurements to achieve the desired accuracy for these measurements.

Additionally, a filter 324 can be configured to filter out light prior to the second detector 323 receiving the light. The filter 324 can be configured to substantially or fully block excitation light wavelength. Additionally, the filter 324 can be configured to allow light that is emitted from the exogenous fluorescent agent to pass therethrough substantially unimpeded. In the illustrated example, the excitation light wavelength can be a blue wavelength and the filter 324 can be configured to allow green light to pass therethrough. As a result, the first detector 322 is configured to measure light received at both the excitation and emission wavelengths, and the second detector 323 is configured to detect light received at the emission wavelength only. Combined with the illumination of the tissues 320 of the patient 104 with light at the excitatory wavelength only and at the emission wavelength only in an alternating series, the measurements from the first detector 322 and a second detector 323 may be analyzed as described in U.S. Pat. Nos. 10,548,521, 10,980,459, 10,952,656, 11,478,172 and 10,194,854 to measure the fluorescence of an exogenous fluorescence agent and to correct the fluorescence measurements by removing the effects of autofluorescence, excitation-wavelength light leak-through and the diffuse reflectance of light according to the correction methods described therein. While the illustrated example only includes a single filter 324, in examples an additional filter can be configured to filter out light prior to the first light detector 322. In other examples, a single filter can be placed before the first light detector 322 rather than the second light detector 323.

The excitation LED 321 (for example, a blue LED) can emit light 325 that is directed toward the exogenous fluorescent agent 112. Additionally, light emitted from the excitation LED 321 can travel through the patient 104 such that the tissue 320 of the patient serves to diffuse the light. The diffuse light can be referred to a diffuse reflectance (DR) signal. Additionally, the light 325 that impacts the exogenous fluorescent agent 112 and the fluorescence emission (Flr) signal 334 travel to the detectors 322, 323. As illustrated, the first detector 322 receives a DR signal 333 labeled as DRex₁ and a Flr signal 332 labeled as Flr₁, and the second detector 323 receives a DR signal 335 labeled as DRex₂ and a Flr signal 334 labeled as Flr₂. The Flr signals 332 and 334 include contributions from the exogenous fluorescent agent 112 and tissue autofluorescence. These measurements are used to arrive at intrinsic fluorescence (IF) signal that is just of the agent as described herein.

FIG. 3 illustrates using a light source 108 in the form of another light emitting diode (LED) 341. In one example, the another LED 341 can be a green LED. In yet other examples, the another LED 341 can be other types of LEDs that provide light at a different wavelength from the excitation LED 321. In one example, the another LED 341 can be operable to emit light in substantially the same wavelength as the emission from the exogenous agent. In other examples, a single LED capable of emitting light at various wavelengths can be implemented. Light emitted from the another LED 341 can travel through the patient 104 such that the tissue 320 of the patient serves to diffuse the light. The diffuse light can be referred to a DR signal, as described herein. As illustrated, the first detector 322 receives a DR signal 342 labeled as DRem₁, and the second detector 323 receives a DR signal 344 labeled as DRem₂. As in FIG. 2, a filter 324 can be implemented. This filter can be the same as in FIG. 2. While the another LED 341 and excitation LED 321 are indicated as being separate from one another, the another LED 341 and excitation LED 321 can be coupled to one another. While the illustrated example only includes a single filter 324, in examples an additional filter can be configured to filter out light prior to the first detector 322. In other examples, a single filter can be placed before the first detector 322 rather than the second detector 323.

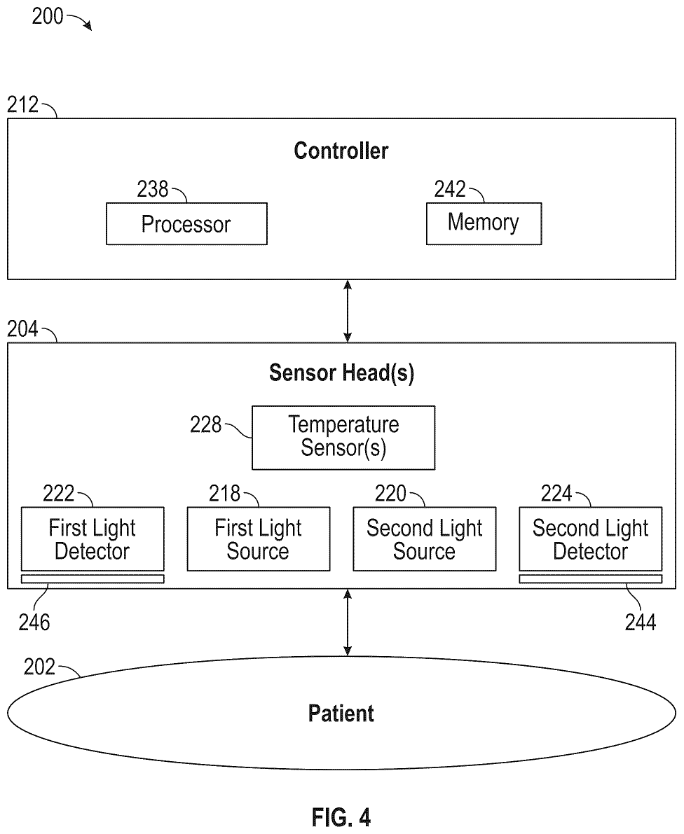

FIG. 4 is an example schematic illustration of an organ monitoring system 200. The system can include a controller 212 that includes a processor 238 and a memory 242. The controller 212 can be coupled to one or more sensor head(s) 204. Each sensor head can include a first light source 218 and a second light source 220. The first light source 218 can be an excitation LED as indicated above. The second light source 220 can be another LED as indicated above. In other examples, the excitation LED can be the second light source 220 and the another LED can be the first light source 218. As illustrated a first light filter 246 and a second light filter 244 is included. In other examples, only one of the first light filter 246 and second light filter 244 can be implemented, such as described in regards to FIGS. 2 and 3. The sensor head 204 can optionally include one or more temperature sensor(s) 228. The one or more temperature sensor(s) 228 can collect data at the same time as the data being collected from the first light detector 222 and/or second light detector 224. The one or more temperature sensor(s) 228 can be used to determine characteristics associated with the first light detector 22 and/or second light detector 224. Additionally, the one or more temperature sensor(s) 228 can be arranged to provide information regarding the patient 202. While the illustrated example includes the controller 212 as separate from the sensor head(s) 204, in other examples the controller 212 and sensor head 204 can be part of a single unit rather than being coupled either wired or wirelessly.

The first light source 218 and second light source 220 can be configured to emit light into the patient 202. The light can be diffused within the patient and a portion of the light is received at the first light detector 222 and/or a portion of the light is received at the second light detector 224. The data obtained by the first light detector 222 and/or the second light detector 224 can be transmitted to the controller 212. The data can be stored in memory 242 or another storage device with which the controller is in electronic communication.

The processor 238 can be operable to execute instructions according to one or more methods as described herein. The processor 238 can be operable to calculate a biological parameter value. In one example, the biological parameter value can be one or more of a GFR and/or RDTC. In other examples, the biological parameter value can be a parameter to describe permeability and/or leaks of the intestinal wall.

In various aspects, the first light source 218 and the second light source 220 can be any light source configured to deliver light at the excitatory wavelength and at the emission wavelength. Typically, the first light source 218 delivers light at an intensity that is sufficient to penetrate the tissues of the patient 202 to the exogenous fluorescent agent with sufficient intensity remaining to induce light at the emission wavelength by the exogenous fluorescent agent. Typically, the first light source 218 delivers light at an intensity that is sufficient to penetrate the tissues of the patient 202 to the exogenous fluorescent agent with sufficient intensity remaining after scattering and/or absorption to induce fluorescence at the emission wavelength by the exogenous fluorescent agent. However, the intensity of light delivered by the first light source 218 is limited to an upper value to prevent adverse effects such as tissue burning, tissue tanning, cell damage, and/or photo-bleaching of the exogenous fluorescent agent and/or the endogenous chromophores in the skin (“auto-fluorescence”).

Similarly, the second light source 220 delivers light at the emission wavelength of the exogenous fluorescent agent at an intensity configured to provide sufficient energy to propagate with scattering and absorption through the first region of the patient and out the second region and third region with sufficient remaining intensity for detection by the first light detector 222 and the second light detector 224, respectively. As with the first light source 218, the intensity of light produced by the second light source 220 is limited to an upper value to prevent the adverse effects such as tissue burning, tissue tanning, cell damage, and/or photo-bleaching of the exogenous fluorescent agent and/or the endogenous chromophores in the skin (“auto-fluorescence”).

In various aspects, the first light source 218 and the second light source 220 can be any light source suitable for use with fluorescent medical imaging systems and devices. Non-limiting examples of suitable light sources include: LEDs, diode lasers, pulsed lasers, continuous waver lasers, xenon arc lamps or mercury-vapor lamps with an excitation filter, lasers, and supercontinuum sources. In one aspect, the first light source 218 and/or the second light source 220 can produce light at a narrow spectral bandwidth suitable for monitoring the concentration of the exogenous fluorescence agent using the method described herein. In another aspect, the first light source 218 and the second light source 220 can produce light at a relatively wide spectral bandwidth.

In one aspect, the selection of intensity of the light produced by the first light source 218 and the second light source 220 by the system 200 can be influenced by any one or more of at least several factors including, but not limited to, the maximum permissible exposure (MPE) for skin exposure to a laser beam according to applicable regulatory standards such as ANSI standard Z136.1. In another aspect, light intensity for the system 200 can be selected to reduce the likelihood of photobleaching of the exogenous fluorescent source and/or other chromophores within the tissues of the patient 202 including, but not limited to: collagen, keratin, elastin, hemoglobin within red blood cells and/or melanin within melanocytes. In yet another aspect, the light intensity for the system 200 can be selected in order to elicit a detectable fluorescence signal from the exogenous fluorescent source within the tissues of the patient 202 and the first light detector 222 and/or second light detector. In yet another aspect, the light intensity for the system 200 can be selected to provide suitably high light energy while reducing power consumption, inhibiting heating/overheating of the first light source 218 and the second light source 220, and/or reducing the exposure time of the patient's skin to light from the first light detector 222 and/or second light detector.

In various aspects, the intensity of the first light source 218 and the second light source 220 can be modulated to compensate any one or more of at least several factors including, but not limited to: individual differences in the concentration of chromophores within the patient 202, such as variation in skin pigmentation. In various other aspects, the detection gain of the light detectors can be modulated to similarly compensate for variation in individual differences in skin properties. In an aspect, the variation in skin pigmentation can be between two different individual patients 202, or between two different positions on the same patient 202. In an aspect, the light modulation can compensate for variation in the optical pathway taken by the light through the tissues of the patient 202. The optical pathway can vary due to any one or more of at least several factors including but not limited to: variation in separation distances between the light sources and light detectors of the system 200; variation in the secure attachment of the sensor head 204 to the skin of the patient 202; variation in the light output of the light sources due to the exposure of the light sources to environmental factors such as heat and moisture; variation in the sensitivity of the light detectors due to the exposure of the light detectors to environmental factors such as heat and moisture; modulation of the duration of illumination by the light sources, and any other relevant operational parameter.

In various aspects, the first light source 218 and the second light source 220 can be configured to modulate the intensity of the light produced as needed according to any one or more of the factors described herein above. In one aspect, if the first light source 218 and the second light source 220 are devices configured to continuously vary output fluence as needed, for example LED light sources, the intensity of the light can be modulated electronically using methods including, but not limited to, modulation of the electrical potential, current, and/or power supplied to the first light source 218 and/or the second light source 220. In another aspect, the intensity of the light can be modulated using optical methods including, but not limited to: partially or fully occluding the light leaving the first light source 218 and the second light source 220 using an optical device including, but not limited to: an iris, a shutter, and/or one or more filters; diverting the path of the light leaving the first light source 218 and the second light source 220 away from the first region of the patient using an optical device including, but not limited to a lenses, a mirror, and/or a prism.

In various aspects, the intensity of the light produced by the first light source 218 and the second light source 220 can be modulated via control of the laser fluence, defined herein as the rate of energy within the produced light beam. In one aspect, the laser fluence can be limited to ranges defined by safety standards including, but not limited to, ANSI standards for exposure to laser energy such as ANSI Z136.1.

In various aspects, the pulse width of the light produced by the first light source 218 and the second light source 220 can be independently selected to be a duration ranging from about 0.0001 seconds to about 0.5 seconds.

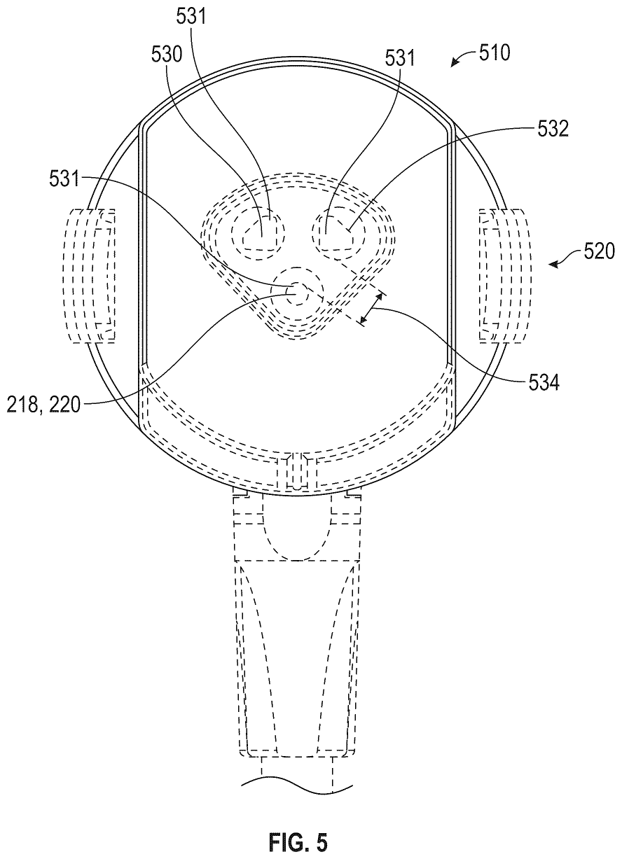

FIG. 5 illustrates an example of a sensor head having one or more light sources and two or more light detectors. As illustrated, a single aperture 531 formed in the sensor head 510 allows light from a first light source 218 and a second light source 220 to pass therethrough. The sensor head 510 also includes a first detector 530 and a second detector 532. Respective apertures 531 can be formed in the sensor head 510 to allow light to reach the first detector 530 and/or the second detector 532. A distance 534 separates the second detector 532 from the one or more light sources 218, 220. The sensor head 510 can also include clip receivers 520 that are designed to be coupled to one or more components not shown.

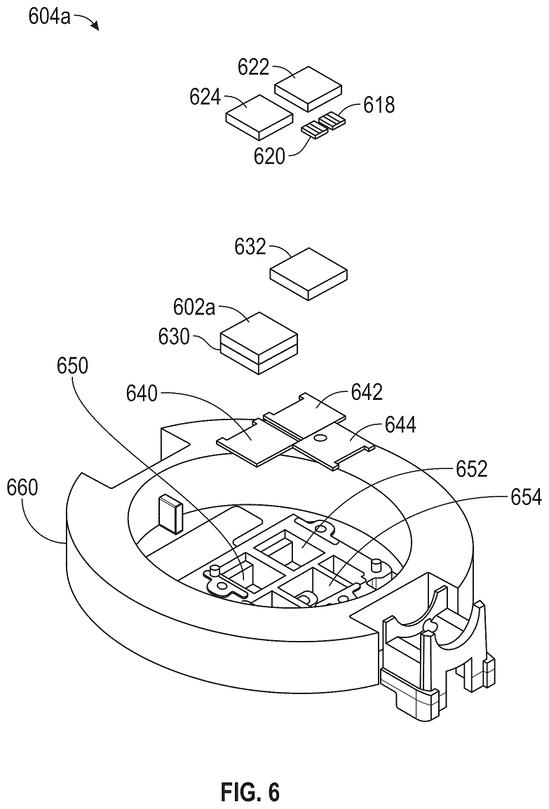

FIG. 6 is an exploded view of an inner housing 660 of the sensor head 510 illustrated in FIG. 5. FIG. 6 is an isometric view of the sensor head 604a with the upper housing and various electrical components removed to expose an inner housing 660. The inner housing 660 is contained within the housing. The inner housing 660 contains a sensor mount with a first detection well 652, a second detection well 650, and a light source well 654 formed therethrough. The first light detector 622 is mounted within the first detection well 652 and the second light detector 624 is mounted within the second detection well 650. The first and second light sources 618/620 are mounted within the light source well 654. In an aspect, the first detection well 652, second detection well 650, and light source well 654 of the sensor mount are optically isolated from one another to ensure that light from the light sources 618/620 does not reach the light detectors 622/624 without coupling through the skin of the patient. The separation between the two detection wells 652/650 ensures that the detected fluorescence signal from the exogenous fluorescent agent is distinguishable from the unfiltered excitation light, as described in detail above.

In one aspect, optically transparent windows 640, 642, and 644 are coupled within first detection aperture, second detection aperture, and light source aperture, respectively, to seal the apertures while also providing optically transparent conduits between the tissues and the interior of the sensor head 604a. In addition, diffusers 630, 632 are coupled over optically transparent windows 640, 642, and 644, respectively. The diffusers 630, 632 are provided to spatially homogenize light delivered to the tissues by light sources 618/620 and to spatially homogenize light detected by light detectors 622/624. In an aspect, the absorption filter 602a is coupled to the diffuser 630. In one aspect, an optically transparent adhesive is used to couple the absorption filter 602a to the diffuser 630.

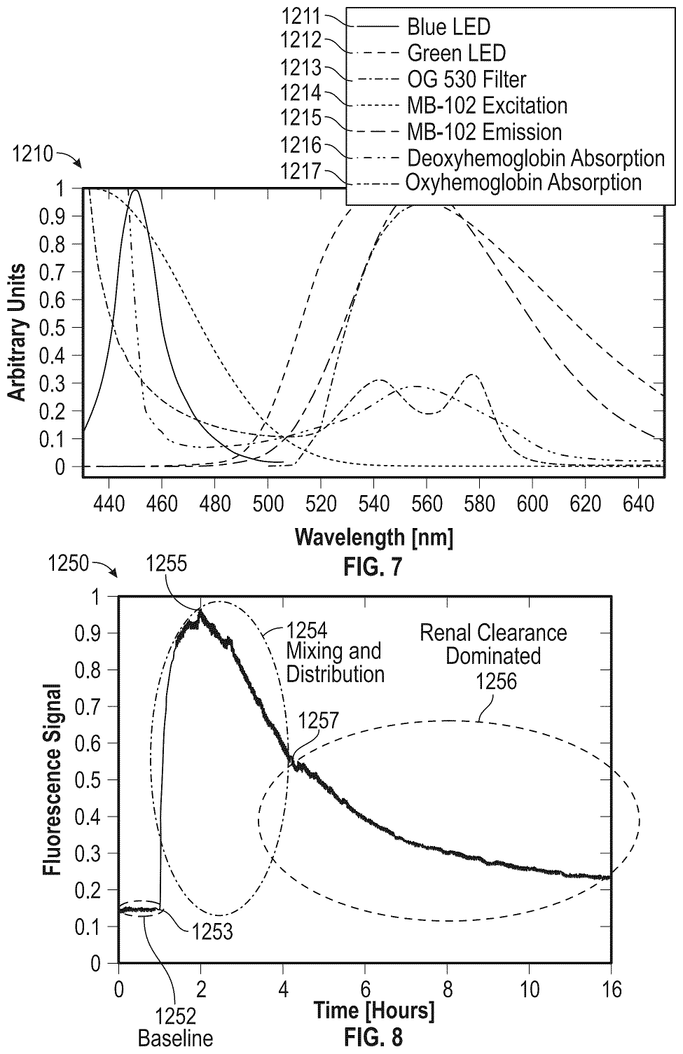

FIG. 7 is a graph 1210 summarizing the absorption, transmission, and emission spectra of various devices, materials, and compounds associated with the non-invasive monitoring of an exogenous fluorescent agent in vivo defined over light wavelengths ranging from about 430 nm to about 650 nm. By way of illustrative example, FIG. 7 is a graph summarizing the absorption spectra for (HbO₂) and (Hb), as well as the absorption (1214) and emission spectra of frequency spectra of relmapirazin (also referred to as MB-102) (1215), an exogenous fluorescent agent in one aspect. Emission spectra for a blue LED light source 1211 and a green LED light source 1212 are also shown superimposed over the other spectra of FIG. 7. In this aspect, the system can include a blue LED as the first light source 218, and the excitatory wavelength for the system can be the isosbestic wavelength of about 450 nm. As shown in FIG. 7, the Hb absorbance spectra is strongly sloped at the isosbestic wavelengths of about 420 nm to about 450 nm, indicating that the relative absorbance of (HbO₂) (1217) and (Hb) (1216) at the isosbestic wavelength of about 450 nm is sensitive to small changes in excitatory wavelength. However, at wavelengths above about 500 nm, the (HbO₂)/(Hb) spectra are less steeply sloped, and a broader band light source including, but not limited to, an LED with a bandpass filter 1213 can suffice for use as a first light source.

In another aspect, the excitatory wavelength can be selected to enhance the contrast in light absorbance between the exogenous fluorescent agent and the chromophores within the tissues of the patient. By way of non-limiting example, as shown in FIG. 7 at the isosbestic wavelength of 452 nm, the light absorption of the relmapirazin is more than three-fold higher than the light absorption of the (HbO₂) and the (Hb). Without being limited to any particular theory, a higher proportion of light illuminating the tissue of the patient at a wavelength of about 450 nm will be absorbed by the relmapirazin relative to the (HbO₂) and (Hb), thus enhancing the efficiency of absorption by the relmapirazin and reducing the intensity of light at the excitatory wavelength needed to elicit a detectable fluorescence signal.

In various aspects, a second isosbestic wavelength can also be selected as the emission wavelength for the system. By way of non-limiting example, FIG. 7 shows an emission spectrum of the relmapirazin exogenous contrast agent (1215) that is characterized by an emission peak at a wavelength of about 550 nm. In this non-limiting example, the isosbestic wavelength of 570 nm can be selected as the emission wavelength to be detected by first and second detectors. In various other aspects, the emission wavelength of the system can be selected to fall within a spectral range characterized by relatively low absorbance of the chromophores within the tissues of the patient 202. Without being limited to any particular theory, the low absorbance of the chromophores at the selected emission wavelength can reduce the losses of light emitted by the exogenous fluorescent agent and enhancing the efficiency of fluorescence detection.

FIG. 8 is a graph 1250 of representative intrinsic fluorescence signal measurements (IF_{agent}) detected by a renal monitoring device obtained before and after administration of an exogenous fluorescent agent. FIG. 8 is a graph of fluorescence measurements obtained from a patient over a period of about 15 hours after administration of an exogenous fluorescence agent such as relmapirazin after a baseline period 1252 of about 1 hour. The pre-administration/baseline period 1252 is characterized by a relatively low and stable fluorescence level, likely due the absence of exogenous fluorescent agent in the blood of the patient. After the administration 1253 of the exogenous fluorescence agent, the fluorescence measurements exhibit a sharp increase to a peak concentration 1255, followed by a relatively smooth exponential decrease back to background fluorescence levels as the kidneys eliminate the exogenous fluorescence agent from the blood of the patient. Without being limited to any particular theory, it is thought the administered exogenous fluorescence agent is likely well-mixed after an amount of time has elapsed in the decrease of the exponential concentration. After an exogenous fluorescent agent, such as relmapirazin, is administered, the exogenous fluorescent agent undergoes an equilibration period of diffusion from the bloodstream into the rest of the extracellular tissues of the patient. Once the diffusion of the exogenous fluorescent agent into the extracellular tissues of the patient reaches a quasi-steady state condition, post-equilibration is achieved and the fluorescence signal can be characterized as a single exponential decay. Without being limited to any particular theory, the pre-equilibration region of the measurement data set is assumed to be characterized as a temporal region of the IF dataset.

As illustrated in FIG. 8, the post-administration period includes a mixing and distribution portion 1254 and a renal clearance dominated portion 1256. The region that the mixing and distribution portion 1254 and the renal clearance dominated portion 1256 touch and/or overlap can be defined as a beginning of a region dominated by single exponential decay 1257. In order to calculate the region dominated by single exponential decay 1257, the present system can implement the following method and apparatus.

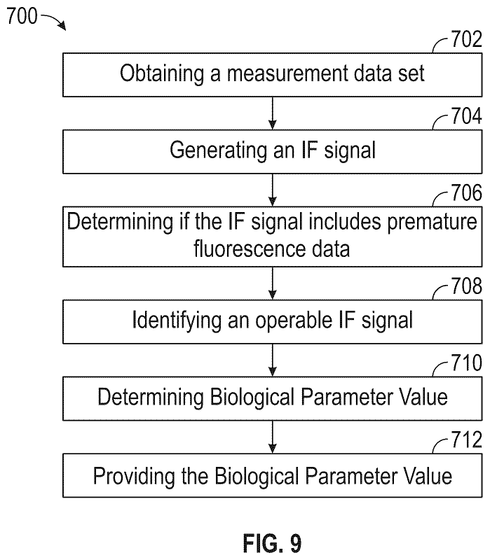

FIG. 9 illustrates a flow chart corresponding to an example method 700. Although the example method 700 depicts a particular sequence of operations, the sequence may be altered without departing from the scope of the present disclosure. For example, some of the operations depicted may be performed in parallel or in a different sequence that does not materially affect the function of the method 700. In other examples, different components of an example device or system that implements the method 700 may perform functions at substantially the same time or in a specific sequence.

According to some examples, as depicted in FIG. 9, the method 700 includes obtaining a measurement data set comprising a plurality of measurement entries that are obtained before and after administration of an exogenous fluorescent agent at block 702. For example, the system and/or apparatus illustrated in FIG. 4 may obtain a measurement data set comprising a plurality of measurement entries that are obtained before and after administration of an exogenous fluorescent agent. For example, the system and/or apparatus can be used to collect data from the one or more sensor heads using the one or more light sources and one or more detectors. The collection of the data can be managed by the controller and/or processor. In at least one example, the controller and/or processor can store the data locally for processing. In another example, the data can be sent to a secure remote computer to perform the processing. The system and/or apparatus can be placed such that the one or more sensor heads are in proximity to or touching the patient from which the data is measured. The system and/or apparatus can be operational for a predetermined period of time prior to administration. One example of the data collection and results of the processing of data to provide a renal function value can be illustrated in FIG. 8.

In at least one example, each of the plurality of measurement entries comprises at least two measurements, one of the at least two measurements being a fluorescence emission (Flr) signal and a second of the at least two measurements being detected at a region adjacent to a diffuse reflecting medium by a filtered light detector during illumination of the diffuse reflecting medium by excitatory-wavelength light. In other examples, the filtered light detector can be such that a filter is placed in front of the light detector to optically filter the light based on a physical filter. In other examples, the light can be filtered by processing of the data from the light detector.

In another example, each of the plurality of measurement entries comprises at least one measurement of the Flr signal detected at the diffuse reflecting medium by a filtered light detector during illumination of the diffuse reflecting medium by excitatory-wavelength light, wherein generating the IF signal includes transforming the at least one measurement according to a transformation relation comprising a mathematical equation converting the Flr signal to the IF signal. In yet another example, each of the plurality of measurement entries comprises at least one measurement signal detected at a diffuse reflecting medium by a filtered light detector during illumination of the diffuse reflecting medium by light with a wavelength similar or equal to the exogenous fluorescent agent wavelength.

According to some examples, the method includes generating an intrinsic fluorescence (IF) signal representing a detected fluorescence intensity emitted by the exogenous fluorescent agent from within a diffuse reflecting medium at block 704. For example, the processor of the system illustrated in FIG. 4 may generate an IF signal representing a detected fluorescence intensity emitted by the exogenous fluorescent agent from within a diffuse reflecting medium. In at least one example, the diffuse reflecting medium is one or more portions of the patient in question having the properties described herein as related to a diffuse reflecting medium. In at least one example, the generation of the IF signal includes directly generating the IF signal from measured data. In some configurations of the system and/or apparatus of FIG. 4, it is possible to directly measure the IF signal. In other configurations, the system and/or apparatus can perform calculations. In other examples as presented herein, the generation of the IF signal can be through additional calculations. In yet other examples, generating the IF signal includes combining the at least two measurements according to a transformation relation comprising a mathematical equation converting the fluorescence emission (Flr) signal to the IF signal. In still yet other examples of methods of generating an IF signal are described in U.S. Pat. Nos. 10,548,521, 10,980,459, 10,952,656, 11,478,172, 10,194,854 and 11,602,570.

According to some examples, the method includes determining if the IF signal includes a portion of premature fluorescence data at block 706. For example, the processor of the system illustrated in FIG. 4 may determine if the IF signal includes a portion of premature fluorescence data. This can be seen in relation to FIG. 8, as one example for determining a renal function value. In at least one example, the method at block 706 of FIG. 9 includes determining a segment of decreasing IF signal. In other examples, in determining a segment of decreasing IF signal, the method can identify a predetermined fitting window. The predetermined fitting window refers to a predetermined time window such that the window has a length of time that is set at a predetermined length. The predetermined time window can be such that the predetermined time window adjusts over time. In other examples, the predetermined time window has a fixed amount of time, but the window itself can move through the data. In one aspect, when determining the biological parameter value for renal function of the patient, the method can further include performing a single exponent curve-fit of the IF signal according to: IF_{fit}=C₀+C₁*e^{-t/RDTC}, where IF_{fit} represents a fit to a portion of IF, C₀ and C₁ are curve-fit constants, t is time, and RDTC is a time parameter. Additionally, the method can calculate a plurality of RDTC values in at least two segments of the predetermined minimum fitting window. In this example, the RDTC is a parameter that can change over time. Furthermore, the method can compare the plurality of RDTC values from the at least two segments until all the plurality of RDTC values correspond to positive values.

In at least one example for determining a renal function value, in determining if the IF signal includes a portion of premature fluorescence data, the method can determine a preliminary GFR value (GFRₚᵣₑₗ) which occurs at a preliminary GFR time (t_{GFRprel}). In one example, determining the GFRₚᵣₑₗ comprises calculating a renal decay time constant (RDTC) by performing an initial estimate of a single exponent curve-fit of the IF signal across at least sequential or overlapping portions of the IF signal, based on a determination of when a segment of the IF signal before t_{GFRprel} surpasses a quality threshold value. The method can also include determining a start time tₛₜₐᵣₜ, which represents a time point prior to evaluation of t_{GFRprel} and during the determined segment of decreasing IF signal; determining a beginning of a fitting interval for the single exponent curve-fit that is a sum of the start time tₛₜₐᵣₜ and a value from a calculation of a first constant multiplied by Euler's number to an exponent added with a second constant (t_{toeq}); setting the fitting interval as the single exponent curve-fit. In one example, tₛₜₐᵣₜ is determined once GFRₚᵣₑₗ has been determined at t_{GFRprel} and tₛₜₐᵣₜ IS t_{GFRprel} minus a time tᵢₙₜₑᵣₚₒₗ which is determined by a linear interpolation between: a low GFR boundary (GFR_{low}) with an associated time t_{low} and/or a high GFR boundary (GFR_{high}) with an associated time t_{high}, using the GFRₚᵣₑₗ.

In a further example, the method can determine a start time (tₛₜₐᵣₜ) which represents a time point prior to the single exponent curve-fit of the operable IF signal range and during the determined segment of decreasing IF signal. In at least one example, the method sets a start point (t_{eq}) of the single exponent curve-fit for the operable IF signal range, wherein t_{eq}=tₛₜₐᵣₜ+t_{roeq}, and t_{toeq}=A×e^{-GFRprel/B}+C with A, B, and C being constants. The method can also set t_{eq} based on a determination of when the single exponent curve-fit according to the IF_{fit} equation surpasses a quality threshold value. The quality threshold value can be a fixed value based on prior measurements. In other examples, the quality threshold value is a set value. In at least one example, the method can determine a peak of IF signal through a recursive filter. The recursive filter can be a variety of different filters. Additionally, the method can exclude data from fitting for a predetermined time after the peak of the IF signal. The amount of data excluded from the fitting can be a fixed amount of predetermined time. In at least one example, the fixed amount of time can be between ten minutes and ten hours. In other examples, the fixed amount of time can be between twenty minutes and five hours. In yet other examples, the fixed amount of time can be between twenty minutes and one and a half hours. In other examples, the amount of data excluded can use a variable amount of time to determine when the data is stable thereby increasing the speed of the remainder of the process.

Furthermore, the method can in determining GFRₚᵣₑₗ determine an estimate of the single exponent curve-fit during the determined segment of decreasing IF signal, and wherein setting the start point of the single exponent curve-fit in the measurement data set that occurs at or after the initial estimate of the single exponent curve-fit of the measurement data set comprises performing a reverse-looking component filter. In this process, a recursive or variable method can be implemented to improve the accuracy of the overall result. The reverse-looking component filter can be implemented to exclude portions of the data set in a reverse looking fashion. That is, the data to be initially excluded from the calculations is determined to be above a threshold then the data can be included. For example, if the data that was excluded is stable, the data can be included thereby decreasing the overall time in the process. However, if the data does not hold the same quality of the present data or data recorded prior to that set of data, the set of data remains excluded.

According to some examples, the method includes identifying, upon determination of the portion of premature fluorescence data, an operable IF signal range by filtering the IF signal associated with premature fluorescence data at block 708 depicted in FIG. 9. For example, the processor of the system illustrated in FIG. 4 may identify, upon determination of the portion of premature fluorescence data, an operable IF signal range by filtering the IF signal associated with premature fluorescence data. In at least one example, the filtering of the IF signal associated with premature fluorescence data includes omitting the portion of premature fluorescence data from the generation of the IF signal. In other examples, the filter of the IF signal can be such that calculations are made to modify the IF signal to more accurately represent the operable IF signal. In at least one example, the filtering of the IF signal associated with premature fluorescence data includes omitting the portion of premature fluorescence data from the identification of the IF signal, such that the portion of premature fluorescence data is included in IF signal and then subsequently excluded from the operable IF signal.

According to some examples, the method includes determining a biological parameter value in the patient based on a rate of change of the operable IF signal at block 710 depicted in FIG. 9. For example, the processor of the system illustrated in FIG. 4 may determine a biological parameter value in the patient based on a rate of change of the operable IF signal. In one example, the biological parameter value is a glomerular filtration rate (GFR). In another example, the biological parameter value is an intestinal wall barrier function.

In at least one example, the method determines the GFR based on the rate of change of the operable IF signal after the beginning of a single exponent curve-fit of the operable IF signal. One example of the single exponent curve-fit is explained herein. In another example, the method of determining the GFR is disclosed in U.S. Pat. No. 11,602,570. In at least one example, determining the GFR value comprises calculating a renal decay time constant (RDTC) by performing a single exponent curve-fit of the operable IF signal across at least sequential portions of the operable IF curve. Sequential portions of the operable IF curve refer to portions that are adjacent to or overlapping one another in time. In one example, the single exponent curve-fit of the operable IF signal comprises log-transforming the operable IF signal and then fitting a linear function to the log-transformed operable IF signal.

The method can, also in determining the GFR value, begin at the start point of the single exponent curve-fit for the operable IF signal (t_{eq}) identified in block 706. Additionally, the method, in determining the GFR, can be based on the rate of change of the operable IF signal after the beginning of the single exponent curve-fit of the operable IF signal.

According to some examples, the method includes providing the biological parameter value at block 712. For example, the processor of system illustrated in FIG. 4 may provide the biological parameter value. In at least one example, the biological parameter value is one of GFR and/or intestinal barrier wall function. In at least one example, providing the biological parameter value includes displaying the biological parameter value on a display screen. In another example, providing the biological parameter value includes transmitting the biological parameter value to a remote device. In yet other examples, the method can include transmitting the biological parameter value to a remote device and also displaying the biological parameter value on a local display screen. The local display screen can be included on the device as illustrated in FIG. 4 above. The local display screen can be included with the controller 212 of the system 200 of FIG. 4.

Illustrative aspects of the disclosure include:

Aspect 1. A method of determining glomerular filtration rate (GFR) using an exogenous fluorescent agent in a patient, the method comprising: obtaining a measurement data set comprising a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; determining if the IF signal includes a portion of premature fluorescence data; identifying, upon determination of the portion of premature fluorescence data, a range of operable IF signals by filtering the IF signal associated with premature fluorescence data; determining a GFR value in the patient based on a rate of change across the operable IF signal range; providing the GFR value.

Aspect 2. The method of Aspect 1, wherein determining if the IF signal includes the portion of premature fluorescence data includes determining a segment of decreasing IF signal.

Aspect 3. The method of Aspect 2, wherein determining if the IF signal includes a portion of premature fluorescence data comprises: determining a preliminary GFR value (GFRₚᵣₑₗ) which occurs at a preliminary GFR time (t_{GFRprel}), wherein determining the GFRₚᵣₑₗ comprises calculating a renal decay time constant (RDTC) by performing an initial estimate of a single exponent curve-fit of the IF signal across at least sequential or overlapping portions of the IF signal,based on a determination of when a segment of the IF signal before t_{GFRprel} surpasses a quality threshold value.

Aspect 4. The method of Aspect 3, further comprising: determining a start time tₛₜₐᵣₜ, which represents a time point prior to evaluation of t_{GFRprel} and during the determined segment of decreasing IF signal; determining a beginning of a fitting interval for the single exponent curve-fit of the operable IF signal that is a sum of the start time tₛₜₐᵣₜ and a value from a calculation of a first constant multiplied by Euler's number to an exponent added with a second constant (t_{toeq}); setting the fitting interval as the single exponent curve-fit of the operable IF signal.

Aspect 5. The method of Aspect 4, wherein tₛₜₐᵣₜ is determined once GFRₚᵣₑₗ has been determined at t_{GFRprel} and tₛₜₐᵣₜ is t_{GFRprel} minus a time tᵢₙₜₑᵣₚₒₗ which is determined by a linear interpolation between: a low GFR boundary (GFR_{low}) with an associated time t_{low} and/or a high GFR boundary (GFR_{high}) with an associated time t_{high}, using the GFRₚᵣₑₗ.

Aspect 6. The method of Aspect 3, further comprising: determining a start time (tₛₜₐᵣₜ) which represents a time point prior to the single exponent curve-fit of the operable IF signal and during the determined segment of decreasing IF signal; setting a start point (t_{eq}) of the single exponent curve-fit of the operable IF signal range; wherein t_{eq}=tₛₜₐᵣₜ+t_{toeq}, and t_{toeq}−A×e^{-GFR}^{_{prel/B}}+C with A, B, and C being constants.

Aspect 7. The method of Aspect 3, wherein determining the segment of decreasing IF signal comprises: identifying a predetermined fitting window; performing a single exponent curve-fit of the IF signal according to: IF_{fit}=C₀+C₁*e^{-t/RDTC}, where IF_{fit} represents a fit to a portion of IF, C₀ and C₁ are curve-fit constants, t is time, and RDTC is a time parameter; calculating RDTC values in at least two segments of a predetermined fitting window; comparing RDTC values from the at least two segments until all the RDTC values correspond to positive values.

Aspect 8. The method of Aspect 7, further comprising: determining a start time (tₛₜₐᵣₜ) which represents a time point prior to the single exponent curve-fit of the operable IF signal and during the determined segment of decreasing IF signal; setting a start point (t_{eq}) of the single exponent curve-fit for the operable IF signal; wherein t_{eq}=tₛₜₐᵣₜ+t_{toeq}, and t_{toeq}−A×e^{-GFRprel/B}+C with A, B, and C being constants.

Aspect 9. The method of Aspect 8, wherein setting t_{eq} is based on a determination of when the single exponent curve-fit according to the IF_{fit} equation surpasses a quality threshold value.

Aspect 10. The method of Aspect 8, wherein tₛₜₐᵣₜ is determined once GFRₚᵣₑₗ has been determined at t_{GFRprel} and tₛₜₐᵣₜ IS t_{GFRprel} minus a time (tᵢₙₜₑᵣₚₒₗ) which is determined by a linear interpolation between: a low GFR boundary (GFR_{low}) with an associated time t_{low} and/or a high GFR boundary (GFR_{high}) with an associated time t_{high}, using the GFRₚᵣₑₗ.

Aspect 11. The method of Aspect 3, wherein determining if the IF signal includes a portion of premature fluorescence data further comprises performing a reverse-looking component filter.

Aspect 12. The method of any one of Aspects 1-11, wherein determining the GFR value comprises calculating a renal decay time constant (RDTC) by performing a single exponent curve-fit of the operable IF signal across at least sequential or overlapping portions of the operable IF signal.

Aspect 13. The method of Aspect 12, wherein the single exponent curve-fit of the operable IF signal comprises log-transforming the operable IF signal and then fitting a linear function to the log-transformed operable IF signal.

Aspect 14. The method of Aspect 12, wherein the determining the GFR value begins at a start point of the single exponent curve-fit for the operable IF signal (t_{eq}).

Aspect 15. The method of Aspect 12, wherein the determining the GFR is based on the rate of change of the operable IF signal after a beginning of the single exponent curve-fit of the operable IF signal.

Aspect 16. The method of any one of Aspects 1-15, wherein providing the GFR value includes displaying the GFR value on a display screen.

Aspect 17. The method of any one of Aspects 1-16, wherein providing the GFR value includes transmitting the GFR value to a remote device.

Aspect 18. The method of any one of Aspects 1-17, wherein generating the IF signal includes directly generating the IF signal from measured data.

Aspect 19. The method of any one of Aspects 1-18, wherein filtering the IF signal associated with premature fluorescence data includes omitting the portion of premature fluorescence data from the generation of the IF signal.

Aspect 20. The method of any one of Aspects 1-19, wherein each of the plurality of measurement entries comprises at least two measurements, one of the at least two measurements being a fluorescence emission (Flr) signal and a second of the at least two measurements being a diffuse reflectance signal detected at a region adjacent to the diffuse reflecting medium by a filtered light detector during illumination of the diffuse reflecting medium by excitatory-wavelength light; wherein generating the IF signal includes combining the at least two measurements according to a transformation relation comprising a mathematical equation converting the Flr signal to the IF signal.

Aspect 21. The method of any one of Aspects 1-20, wherein each of the plurality of measurement entries comprises at least one measurement of a fluorescence emission (Flr) signal detected at the diffuse reflecting medium by a filtered light detector during illumination of the diffuse reflecting medium by excitatory-wavelength light; wherein generating the IF signal includes transforming the at least one measurement according to a transformation relation comprising a mathematical equation converting the Flr signal to the IF signal.

Aspect 22. The method of Aspect 21, wherein the excitatory-wavelength light comprises a wavelength of the exogenous fluorescent agent.

Aspect 23. A method of determining a biological parameter using an exogenous fluorescent agent in a patient, the method comprising: obtaining a measurement data set comprising a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; determining if the IF signal includes a portion of premature fluorescence data; identifying, upon determination of the portion of premature fluorescence data, an operable IF signal range by filtering the IF signal associated with premature fluorescence data; determining a biological parameter value in the patient based on a rate of change of the operable IF signal; providing the biological parameter value.

Aspect 24. A method of determining glomerular filtration rate (GFR) using an exogenous fluorescent agent in a patient, the method comprising: obtaining a measurement data set comprising a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; determining if the IF signal includes a portion of premature fluorescence data; identifying, upon determination of the portion of premature fluorescence data, a range of operable IF signals by filtering the IF signal associated with premature fluorescence data; determining a GFR value in the patient based on a rate of change across the operable IF signal range; providing the GFR value.

Aspect 25. The method of Aspect 24, wherein determining if the IF signal includes the portion of premature fluorescence data includes determining a segment of decreasing IF signal.

Aspect 26. The method of Aspect 25, wherein determining if the IF signal includes a portion of premature fluorescence data comprises: determining a preliminary GFR value (GFRₚᵣₑₗ) which occurs at a preliminary GFR time (t_{GFRprel}), wherein determining the GFRₚᵣₑₗ comprises calculating a renal decay time constant (RDTC) by performing an initial estimate of a single exponent curve-fit of the IF signal across at least sequential or overlapping portions of the IF signal,based on a determination of when a segment of the IF signal before t_{GFRprel} surpasses a quality threshold value.

Aspect 27. The method of Aspect 26, further comprising: determining a start time tₛₜₐᵣₜ, which represents a time point prior to evaluation of t_{GFRprel} and during the determined segment of decreasing IF signal; determining a beginning of a fitting interval for the single exponent curve-fit of the operable IF signal that is a sum of the start time tₛₜₐᵣₜ and a value from a calculation of a first constant multiplied by Euler's number to an exponent added with a second constant (t_{toeq}); setting the fitting interval as the single exponent curve-fit of the operable IF signal.

Aspect 28. The method of Aspect 27, wherein tₛₜₐᵣₜ is determined once GFRₚᵣₑₗ has been determined at t_{GFRprel} and tₛₜₐᵣₜ is t_{GFRprel} minus a time tᵢₙₜₑᵣₚₒₗ which is determined by a linear interpolation between: a low GFR boundary (GFR_{low}) with an associated time t_{low} and/or a high GFR boundary (GFR_{high}) with an associated time t_{high}, using the GFRₚᵣₑₗ.

Aspect 29. The method of Aspect 26, further comprising: determining a start time (tₛₜₐᵣₜ) which represents a time point prior to the single exponent curve-fit of the operable IF signal and during the determined segment of decreasing IF signal; setting a start point (t_{eq}) of the single exponent curve-fit of the operable IF signal range; wherein t_{eq}=tₛₜₐᵣₜ+t_{toeq}, and t_{toeq}−A×e^{-GFRprel/B}+C with A, B, and C being constants.

Aspect 30. The method of Aspect 26, wherein determining the segment of decreasing IF signal comprises: identifying a predetermined fitting window; performing a single exponent curve-fit of the IF signal according to: IF_{fit}=C₀+C₁*e^{-t/RDTC}, where IF_{fit} represents a fit to a portion of IF, C₀ and C₁ are curve-fit constants, t is time, and RDTC is a time parameter; calculating RDTC values in at least two segments of a predetermined fitting window; comparing RDTC values from the at least two segments until all the RDTC values correspond to positive values.

Aspect 31. The method of Aspect 30, further comprising: determining a start time (tₛₜₐᵣₜ) which represents a time point prior to the single exponent curve-fit of the operable IF signal and during the determined segment of decreasing IF signal; setting a start point (t_{eq}) of the single exponent curve-fit for the operable IF signal; wherein t_{eq}=tₛₜₐᵣₜ+t_{toeq}, and t_{toeq}−A×e^{-GFRprel/B}+C with A, B, and C being constants.

Aspect 32. The method of Aspect 31, wherein setting t_{eq} is based on a determination of when the single exponent curve-fit according to the IF_{fit} equation surpasses a quality threshold value.

Aspect 33. The method of Aspect 31, wherein tₛₜₐᵣₜ is determined once GFRₚᵣₑₗ has been determined at t_{GFRprel} and tₛₜₐᵣₜ is t_{GFRprel} minus a time (tᵢₙₜₑᵣₚₒₗ) which is determined by a linear interpolation between: a low GFR boundary (GFR_{low}) with an associated time t_{low} and/or a high GFR boundary (GFR_{high}) with an associated time t_{high}, using the GFRₚᵣₑₗ.

Aspect 34. The method of Aspect 26, wherein determining if the IF signal includes a portion of premature fluorescence data further comprises performing a reverse-looking component filter.

Aspect 35. The method of any one of Aspects 24-34, wherein determining the GFR value comprises calculating a renal decay time constant (RDTC) by performing a single exponent curve-fit of the operable IF signal across at least sequential or overlapping portions of the operable IF signal.

Aspect 36. The method of Aspect 35, wherein the single exponent curve-fit of the operable IF signal comprises log-transforming the operable IF signal and then fitting a linear function to the log-transformed operable IF signal.

Aspect 37. The method of Aspect 35, wherein the determining the GFR value begins at a start point of the single exponent curve-fit for the operable IF signal (t_{eq}).

Aspect 38. The method of Aspect 35, wherein the determining the GFR is based on the rate of change of the operable IF signal after a beginning of the single exponent curve-fit of the operable IF signal.

Aspect 39. The method of any one of Aspects 24-38, wherein providing the GFR value includes displaying the GFR value on a display screen.

Aspect 40. The method of any one of Aspects 24-39, wherein providing the GFR value includes transmitting the GFR value to a remote device.

Aspect 41. The method of any one of Aspects 24-40, wherein generating the IF signal includes directly generating the IF signal from measured data.

Aspect 42. The method of any one of Aspects 24-41, wherein filtering the IF signal associated with premature fluorescence data includes omitting the portion of premature fluorescence data from the generation of the IF signal.

Aspect 43. The method of any one of Aspects 24-42, wherein each of the plurality of measurement entries comprises at least two measurements, one of the at least two measurements being a fluorescence emission (Flr) signal and a second of the at least two measurements being a diffuse reflectance signal detected at a region adjacent to the diffuse reflecting medium by a filtered light detector during illumination of the diffuse reflecting medium by excitatory-wavelength light; wherein generating the IF signal includes combining the at least two measurements according to a transformation relation comprising a mathematical equation converting the Flr signal to the IF signal.

Aspect 44. The method of any one of Aspects 24-43, wherein each of the plurality of measurement entries comprises at least one measurement of a fluorescence emission (Flr) signal detected at the diffuse reflecting medium by a filtered light detector during illumination of the diffuse reflecting medium by excitatory-wavelength light; wherein generating the IF signal includes transforming the at least one measurement according to a transformation relation comprising a mathematical equation converting the Flr signal to the IF signal.

Aspect 45. The method of Aspect 44, wherein the excitatory-wavelength light comprises a wavelength of the exogenous fluorescent agent.

Aspect 46. A method of determining a biological parameter using an exogenous fluorescent agent in a patient, the method comprising: obtaining a measurement data set comprising a plurality of measurement entries that are obtained before and after administration of an exogenous fluorescent agent; generating an intrinsic fluorescence (IF) signal representing a detected fluorescence intensity emitted by the exogenous fluorescent agent from within a diffuse reflecting medium; determining if the IF signal includes a portion of premature fluorescence data; identifying, upon determination of the portion of premature fluorescence data, an operable IF signal range by filtering the IF signal associated with premature fluorescence data; determining a biological parameter value in the patient based on a rate of change of the operable IF signal; providing the biological parameter value.

Aspect 47. A system of determining glomerular filtration rate (GFR) using an exogenous fluorescent agent in a patient, the system comprising: at least one storage including a memory configured to store instructions; a processor, configured to execute the instructions and cause the processor to: obtain a measurement data set comprising a plurality of measurement entries that are obtained before and after administration of an exogenous fluorescent agent; generate an intrinsic fluorescence (IF) signal representing a detected fluorescence intensity emitted by the exogenous fluorescent agent from within a diffuse reflecting medium; determine if the IF signal includes a portion of premature fluorescence data; identify, upon determination of the portion of premature fluorescence data, a range of operable IF signals by filtering the IF signal associated with premature fluorescence data; determine a GFR value in the patient based on a rate of change across the operable IF signal range; provide the GFR value.

Aspect 48. The system of Aspect 47, wherein determining if the IF signal includes the portion of premature fluorescence data includes determining a segment of decreasing IF signal.

Aspect 49. The system of Aspect 48, wherein determining if the IF signal includes a portion of premature fluorescence data comprises: determining a preliminary GFR value (GFRₚᵣₑₗ) which occurs at a preliminary GFR time (t_{GFRprel}), wherein determining the GFRₚᵣₑₗ comprises calculating a renal decay time constant (RDTC) by performing an initial estimate of a single exponent curve-fit of the IF signal across at least sequential or overlapping portions of the IF signal,based on a determination of when a segment of the IF signal before t_{GFRprel} surpasses a quality threshold value.

Aspect 50. The system of Aspect 49, wherein the processor is configured to: determine a start time tₛₜₐᵣₜ, which represents a time point prior to evaluation of t_{GFRprel} and during the determined segment of decreasing IF signal; determine a beginning of a fitting interval for the single exponent curve-fit of the operable IF signal that is a sum of the start time tₛₜₐᵣₜ and a value from a calculation of a first constant multiplied by Euler's number to an exponent added with a second constant (t_{toeq}); setting the fitting interval as the single exponent curve-fit of the operable IF signal.

Aspect 51. The system of Aspect 50, wherein tₛₜₐᵣₜ is determined once GFRₚᵣₑₗ has been determined at t_{GFRprel} and tₛₜₐᵣt is t_{GFRprel} minus a time tᵢₙₜₑᵣₚₒₗ which is determined by a linear interpolation between: a low GFR boundary (GFR_{low}) with an associated time t_{low} and/or a high GFR boundary (GFR_{high}) with an associated time t_{high}, using the GFRₚᵣₑₗ.

Aspect 52. The system of Aspect 49, the processor is configured to: determine a start time (tₛₜₐᵣₜ) which represents a time point prior to the single exponent curve-fit of the operable IF signal and during the determined segment of decreasing IF signal; set a start point (t_{eq}) of the single exponent curve-fit of the operable IF signal range; wherein t_{eq}=tₛₜₐᵣₜ+t_{toeq}, and t_{toeq}=A×e^{-GFRprel/B}+C with A, B, and C being constants.

Aspect 53. The system of Aspect 49, wherein determining the segment of decreasing IF signal comprises: identifying a predetermined fitting window; performing a single exponent curve-fit of the IF signal according to: IF_{fit}=C₀+C₁*e^{-t/RDTC}, where IF_{fit} represents a fit to a portion of IF, C₀ and C₁ are curve-fit constants, t is time, and RDTC is a time parameter; calculating RDTC values in at least two segments of a predetermined fitting window; comparing RDTC values from the at least two segments until all the RDTC values correspond to positive values.

Aspect 54. The system of Aspect 53, the processor is configured to: determine a start time (tₛₜₐᵣₜ) which represents a time point prior to the single exponent curve-fit of the operable IF signal and during the determined segment of decreasing IF signal; set a start point (t_{eq}) of the single exponent curve-fit for the operable IF signal; wherein t_{eq}=tₛₜₐᵣₜ+t_{toeq}, and t_{toeq}=A×e^{-GFRprel/B}+C with A, B, and C being constants.

Aspect 55. The system of Aspect 54, wherein setting t_{eq} is based on a determination of when the single exponent curve-fit according to the IF_{fit} equation surpasses a quality threshold value.

Aspect 56. The system of Aspect 54, wherein tₛₜₐᵣₜ is determined once GFRₚᵣₑₗ has been determined at t_{GFRprel} and tₛₜₐᵣₜ is t_{GFRprel} minus a time (tᵢₙₜₑᵣₚₒₗ) which is determined by a linear interpolation between: a low GFR boundary (GFR_{low}) with an associated time t_{low} and/or a high GFR boundary (GFR_{high}) with an associated time t_{high}, using the GFRₚᵣₑₗ.

Aspect 57. The system of Aspect 49, wherein determining if the IF signal includes a portion of premature fluorescence data further comprises performing a reverse-looking component filter.

Aspect 58. The system of any one of Aspects 47-57, wherein determining the GFR value comprises calculating a renal decay time constant (RDTC) by performing a single exponent curve-fit of the operable IF signal across at least sequential or overlapping portions of the operable IF signal.

Aspect 59. The system of Aspect 58, wherein the single exponent curve-fit of the operable IF signal comprises log-transforming the operable IF signal and then fitting a linear function to the log-transformed operable IF signal.

Aspect 60. The system of Aspect 58, wherein the determining the GFR value begins at a start point of the single exponent curve-fit for the operable IF signal (t_{eq}).

Aspect 61. The system of Aspect 58, wherein the determining the GFR is based on the rate of change of the operable IF signal after a beginning of the single exponent curve-fit of the operable IF signal.

Aspect 62. The system of any one of Aspects 47-61, wherein providing the GFR value includes displaying the GFR value on a display screen.

Aspect 63. The system of any one of Aspects 47-62, wherein providing the GFR value includes transmitting the GFR value to a remote device.

Aspect 64. The system of any one of Aspects 47-62, wherein generating the IF signal includes directly generating the IF signal from measured data.

Aspect 65. The system of any one of Aspects 47-64, wherein filtering the IF signal associated with premature fluorescence data includes omitting the portion of premature fluorescence data from the generation of the IF signal.

Aspect 66. The system of any one of Aspects 47-65, wherein each of the plurality of measurement entries comprises at least two measurements, one of the at least two measurements being a fluorescence emission (Flr) signal and a second of the at least two measurements being a diffuse reflectance signal detected at a region adjacent to the diffuse reflecting medium by a filtered light detector during illumination of the diffuse reflecting medium by excitatory-wavelength light; wherein generating the IF signal includes combining the at least two measurements according to a transformation relation comprising a mathematical equation converting the Flr signal to the IF signal.

Aspect 67. The system of any one of Aspects 47-66, wherein each of the plurality of measurement entries comprises at least one measurement of a fluorescence emission (Flr) signal detected at the diffuse reflecting medium by a filtered light detector during illumination of the diffuse reflecting medium by excitatory-wavelength light; wherein generating the IF signal includes transforming the at least one measurement according to a transformation relation comprising a mathematical equation converting the Flr signal to the IF signal.

Aspect 68. The system of Aspect 67, wherein the excitatory-wavelength light comprises a wavelength of the exogenous fluorescent agent.

Aspect 69. A system for determining a biological parameter using an exogenous fluorescent agent in a patient, the system comprising: at least one storage including a memory configured to store instructions; a processor, configured to execute the instructions and cause the processor to: obtain a measurement data set comprising a plurality of measurement entries that are obtained before and after administration of an exogenous fluorescent agent; generate an intrinsic fluorescence (IF) signal representing a detected fluorescence intensity emitted by the exogenous fluorescent agent from within a diffuse reflecting medium; determine if the IF signal includes a portion of premature fluorescence data; identify, upon determination of the portion of premature fluorescence data, an operable IF signal range by filtering the IF signal associated with premature fluorescence data; determine a biological parameter value in the patient based on a rate of change of the operable IF signal; provide the biological parameter value.

What is claimed is:

1. A method of determining glomerular filtration rate (GFR) using an exogenous fluorescent agent in a patient, the method comprising:
obtaining a measurement data set comprising a plurality of measurement entries that are obtained before and after administration of an exogenous fluorescent agent;
generating an intrinsic fluorescence (IF) signal representing a detected fluorescence intensity emitted by the exogenous fluorescent agent from within a diffuse reflecting medium;
determining if the IF signal includes a portion of premature fluorescence data;
identifying, upon determination of the portion of premature fluorescence data, a range of operable IF signals by filtering the IF signal associated with premature fluorescence data;
determining a GFR value in the patient based on a rate of change across the operable IF signal range;
providing the GFR value.

2. The method of claim 1, wherein determining if the IF signal includes the portion of premature fluorescence data includes determining a segment of decreasing IF signal.

3. The method of claim 2, wherein determining if the IF signal includes a portion of premature fluorescence data comprises:
determining a preliminary GFR value (GFRₚᵣₑₗ) which occurs at a preliminary GFR time (t_{GFRprel}), wherein determining the GFRₚᵣₑₗ comprises calculating a renal decay time constant (RDTC) by performing an initial estimate of a single exponent curve-fit of the IF signal across at least sequential or overlapping portions of the IF signal,based on a determination of when a segment of the IF signal before t_{GFRprel} surpasses a quality threshold value.

4. The method of claim 3, further comprising:
determining a start time tₛₜₐᵣₜ, which represents a time point prior to evaluation of t_{GFRprel} and during the determined segment of decreasing IF signal;
determining a beginning of a fitting interval for the single exponent curve-fit of the operable IF signal that is a sum of the start time tₛₜₐᵣₜ and a value from a calculation of a first constant multiplied by Euler's number to an exponent added with a second constant (t_{toeq});
setting the fitting interval as the single exponent curve-fit of the operable IF signal.

5. The method of claim 4, wherein tₛₜₐᵣₜ is determined once GFRₚᵣₑₗ has been determined at t_{GFRprel} and tₛₜₐᵣₜ is t_{GFRprel} minus a time tᵢₙₜₑᵣₚₒₗ which is determined by a linear interpolation between:
a low GFR boundary (GFR_{low}) with an associated time t_{low} and/or a high GFR boundary (GFR_{high}) with an associated time t_{high}, using the GFRₚᵣₑₗ.

6. The method of claim 3, further comprising:
determining a start time (tₛₜₐᵣₜ) which represents a time point prior to the single exponent curve-fit of the operable IF signal and during the determined segment of decreasing IF signal;
setting a start point (t_{eq}) of the single exponent curve-fit of the operable IF signal range;
wherein t_{eq}=tₛₜₐᵣₜ+t_{toeq}, and t_{toeq}=A×e^{-GFRprel/B}+C with A, B, and C being constants.

7. The method of claim 3, wherein determining the segment of decreasing IF signal comprises:
identifying a predetermined fitting window;
performing a single exponent curve-fit of the IF signal according to:
IF_{fit}=C₀+C₁*e^{-t/RDTC}
where IF_{fit} represents a fit to a portion of IF, C₀ and C₁ are curve-fit constants, t is time, and RDTC is a time parameter;
calculating RDTC values in at least two segments of a predetermined fitting window;
comparing RDTC values from the at least two segments until all the RDTC values correspond to positive values.

8. The method of claim 7, further comprising:
determining a start time (tₛₜₐᵣₜ) which represents a time point prior to the single exponent curve-fit of the operable IF signal and during the determined segment of decreasing IF signal;
setting a start point (t_{eq}) of the single exponent curve-fit for the operable IF signal;
wherein t_{eq}=tₛₜₐᵣₜ+t_{toeq}, and t_{toeq}=A×e^{-GFRprel/B}+C with A, B, and C being constants.

9. The method of claim 8, wherein setting t_{eq} is based on a determination of when the single exponent curve-fit according to the IF_{fit} equation surpasses a quality threshold value.

10. The method of claim 8, wherein tₛₜₐᵣₜ is determined once GFRₚᵣₑₗ has been determined at t_{GFRprel} and tₛₜₐᵣₜ is t_{GFRprel} minus a time (tᵢₙₜₑᵣₚₒₗ) which is determined by a linear interpolation between: a low GFR boundary (GFR_{low}) with an associated time t_{low} and/or a high GFR boundary (GFR_{high}) with an associated time t_{high}, using the GFRₚᵣₑₗ.

11. The method of claim 3, wherein determining if the IF signal includes a portion of premature fluorescence data further comprises performing a reverse-looking component filter.

12. The method of claim 1, wherein determining the GFR value comprises calculating a renal decay time constant (RDTC) by performing a single exponent curve-fit of the operable IF signal across at least sequential or overlapping portions of the operable IF signal.

13. The method of claim 12, wherein the single exponent curve-fit of the operable IF signal comprises log-transforming the operable IF signal and then fitting a linear function to the log-transformed operable IF signal.

14. The method of claim 12, wherein the determining the GFR value begins at a start point of the single exponent curve-fit for the operable IF signal (t_{eq}).

15. The method of claim 12, wherein the determining the GFR is based on the rate of change of the operable IF signal after a beginning of the single exponent curve-fit of the operable IF signal.

16. The method of claim 1, wherein providing the GFR value includes displaying the GFR value on a display screen.

17. The method of claim 1, wherein providing the GFR value includes transmitting the GFR value to a remote device.

18. The method of claim 1, wherein generating the IF signal includes directly generating the IF signal from measured data.

19. The method of claim 1, wherein filtering the IF signal associated with premature fluorescence data includes omitting the portion of premature fluorescence data from the generation of the IF signal.

20. The method of claim 1, wherein each of the plurality of measurement entries comprises at least two measurements, one of the at least two measurements being a fluorescence emission (Flr) signal and a second of the at least two measurements being a diffuse reflectance signal detected at a region adjacent to the diffuse reflecting medium by a filtered light detector during illumination of the diffuse reflecting medium by excitatory-wavelength light;
wherein generating the IF signal includes combining the at least two measurements according to a transformation relation comprising a mathematical equation converting the Flr signal to the IF signal.

21. The method of claim 1, wherein each of the plurality of measurement entries comprises at least one measurement of a fluorescence emission (Flr) signal detected at the diffuse reflecting medium by a filtered light detector during illumination of the diffuse reflecting medium by excitatory-wavelength light;
wherein generating the IF signal includes transforming the at least one measurement according to a transformation relation comprising a mathematical equation converting the Flr signal to the IF signal.

22. The method of claim 21, wherein the excitatory-wavelength light comprises a wavelength of the exogenous fluorescent agent.

23. A method of determining a biological parameter using an exogenous fluorescent agent in a patient, the method comprising:
obtaining a measurement data set comprising a plurality of measurement entries that are obtained before and after administration of an exogenous fluorescent agent;
generating an intrinsic fluorescence (IF) signal representing a detected fluorescence intensity emitted by the exogenous fluorescent agent from within a diffuse reflecting medium;
determining if the IF signal includes a portion of premature fluorescence data;
identifying, upon determination of the portion of premature fluorescence data, an operable IF signal range by filtering the IF signal associated with premature fluorescence data;
determining a biological parameter value in the patient based on a rate of change of the operable IF signal;
providing the biological parameter value.
